(19) United States
(12) Patent Application Publication
(10) Pub. No.: US 2019/0014987 A1
(43) Pub. Date: Jan. 17, 2019

(54) OCT CATHETER CONNECTOR STRUCTURE AND MEDICAL IMAGING SYSTEM

(71) Applicant: Sumitomo Electric Industries, Ltd., Osaka-shi (JP)

(72) Inventors: Dai Sasaki, Yokohama-shi (JP); Yoshikyo Tamekuni, Yokohama-shi (JP); Takahiro Kikuchi, Yokohama-shi (JP); Takuro Watanabe, Yokohama-shi (JP)

(21) Appl. No.: 16/069,928

(22) PCT Filed: Jan. 10, 2017
(86) PCT No.: PCT/JP2017/000428

§ 371 Date: Jul. 13, 2018

(30) Foreign Application Priority Data
Jan. 21, 2016 (JP) 2016-009625

(51) Int. Cl. A61B 5/00; G02B 6/38

(52) U.S. Cl. CPC A61B 5/0084; A61B 5/6852; A61B 2562/228; G02B 6/3871; G02B 6/3825; A61B 5/0066

(57) ABSTRACT

A connector structure of an OCT catheter includes an outer casing that has an inner space extending in an axial direction and that has an opening at one end in the axial direction, a connector that is disposed in the inner space so as to be rotatable around the axis in a state in which a connection end is oriented toward the opening, and a stopper that is disposed in the inner space and that restricts rotation of the connector relative to the outer casing. When a part of the outer casing including the one end is pushed into a connection port of a rotational driving device that performs rotational driving, the connector is connectable to an adapter of the rotational driving device; and, when the outer casing is pushed into the connection port, restriction on rotation of the connector by the stopper is removed.

### TECHNICAL FIELD

The present invention relates to a connector structure of an OCT catheter and a medical imaging system.

### BACKGROUND ART

International Publication No. 2009/154103 describes an optical coherence tomography (OCT) apparatus which has a structure for connecting a connector device of a catheter and an adapter device of a driving device, for performing a scanning operation and a pullback operation of an imaging core in the catheter, to each other. The connector device includes a connector fixing member having a hollow cylindrical shape. A projection is formed on a part of the outer surface of the connector fixing member in the longitudinal direction. The adapter device includes an adapter fixing member having a hollow cylindrical shape. An inclined end surface is formed on the adapter fixing member. A cutout portion is formed in a part of the inclined end surface so that the projection of the connector fixing member can be fitted into the cutout portion.

In general, with an OCT catheter, an optical connector disposed in a shell is connected to an optical adapter disposed in a rotational driving device in a state in which the rotational positions thereof are aligned with each other. However, the optical connector is rotatable in the shell. Therefore, it is necessary for a user to connect the optical connector and the optical adapter while checking the rotational position of the optical connector in the shell.

With the existing technology described in International Publication No. 2009/154103, when the connector fixing member is inserted into the adapter fixing member, the projection slides along the inclined end surface, and thereby the rotational position of the connector fixing member is adjusted. However, in general, it is presupposed that the adapter device, including the adapter fixing member, is repeatedly used. In this case, the projection and the inclined surface repeatedly slide over each other, and thereby the inclined surface may deteriorate and may become incapable of sliding smoothly. Moreover, depending on the insertion speed of the connector fixing member, an unnecessary load may be applied to the adapter fixing member, and these fixing members may break.

### SUMMARY OF INVENTION

### Technical Problem

An object of the present invention is to provide a connector structure of an OCT catheter with which it is possible to connect a connector and an adapter easily without directly checking the rotational position of the connector in a shell.

### Solution to Problem

The present invention relates to a connector structure of an OCT catheter to be connected to a rotational driving device that includes an adapter performing rotational driving. The connector structure of an OCT catheter according to the present invention includes an outer casing that has an inner space extending in a direction of an axis and that has an opening at one end thereof in the direction of the axis; a connector that is disposed in the inner space so as to be rotatable around the axis in a state in which a connection end thereof is oriented toward the opening; and a stopper that is disposed in the inner space and that restricts rotation of the connector relative to the outer casing. In the connector structure of an OCT catheter according to the present invention, when a part of the outer casing including the one end is pushed into a connection portion of the rotational driving device, the connector is configured to be connectable to the adapter, and restriction on rotation of the connector by the stopper is removed.

The connector structure may be configured as follows: a mechanism for restricting rotation of the connector by the stopper includes an engaging portion formed in the stopper and an engageable portion formed in the connector, the engageable portion being engageable with the engaging portion; and, when the outer casing is pushed into the connection portion, the stopper moves relative to the outer casing in a direction that is the direction of the axis and in which the stopper is pushed, and the engaging portion and the engageable portion are configured to disengage due to relative movement of the stopper. The connector structure may be configured as follows: the stopper includes an engaging portion and is configured to move in the direction of the axis relative to the outer casing; and the connector includes an engageable portion that is engageable with the engaging portion, and the connector is configured so that, when the stopper moves relative to the outer casing in a direction that is the direction of the axis and in which the stopper is pushed, the engaging portion and the engageable portion are configured to disengage and the connector is configured to rotate relative to the stopper.

The connector structure may be configured as follows: change in position of the stopper relative to the outer casing is limited by an elastic member; and, when the outer casing is pushed into the connection portion, a position of the stopper relative to the outer casing is changed while the elastic member is being deformed, and restriction on rotation of the connector by the stopper is removed. The connector structure may be configured as follows: the connector structure of an OCT catheter includes an elastic member that is disposed between the outer casing and the stopper and that is configured to extend or contract in the direction of the axis; when the elastic member extends and the stopper moves relative to the outer casing in a direction that is the direction of the axis and in which the stopper is pulled out, rotation of the connector is restricted; and, when the elastic portion contracts and the stopper moves relative to the outer casing in a direction that is the direction of the axis and in which the stopper is pushed, restriction on rotation is removed.

The connector structure may be configured as follows: rotation of the stopper relative to the outer casing around the axis is restricted. The connector structure may be configured as follows: the outer casing includes a positioning groove in an inner surface thereof that defines the inner space, the positioning groove being parallel to the axis, and the stopper includes a positioning protrusion that engages with the positioning groove.

A medical imaging system according to the present invention includes the connector structure of an OCT catheter described above and the rotational driving device, the medical imaging system is controlled so that a rotational position of the adapter of the rotational driving device when the outer casing is pushed into the connection portion coincides with a rotational position of the adapter when the outer casing is removed from the connection portion.

### Advantageous Effects of Invention

With the connector structure of an OCT catheter according to the present invention, it is possible to connect a connector and an adapter easily without directly checking the rotational position of the connector in a shell.

### BRIEF DESCRIPTION OF DRAWINGS

FIG. 1 is a conceptual view of an OCT apparatus according to an embodiment.

FIG. 2 is an exploded perspective view of a connector structure of an OCT catheter according to an embodiment of the present invention.

FIG. 3 is a sectional view of the connector structure of an OCT catheter according to the embodiment of the present invention.

FIG. 4 is a perspective view of a stopper of the connector structure of an OCT catheter according to the embodiment of the present invention.

FIG. 5 is a perspective view of a connector of the connector structure of an OCT catheter according to the embodiment of the present invention.

FIGS. 6A and 6B illustrate an operation of connecting an OCT catheter according to the embodiment of the present invention.

### DESCRIPTION OF EMBODIMENTS

Hereinafter, an embodiment according to the present invention will be described in detail with reference to the drawings. For convenience, elements that are substantially the same will be denoted by the same numerals, and redundant description thereof may be omitted. In the following description, the axial direction (longitudinal direction) of an OCT catheter will be referred to as the “front-back direction”, the proximal side will be referred to as the “front side”, and the distal side will be referred to as the “rear side”. Unless otherwise noted, the terms “radial direction”, “circumferential direction”, and “rotation” are used with respect to the axis of an OCT catheter.

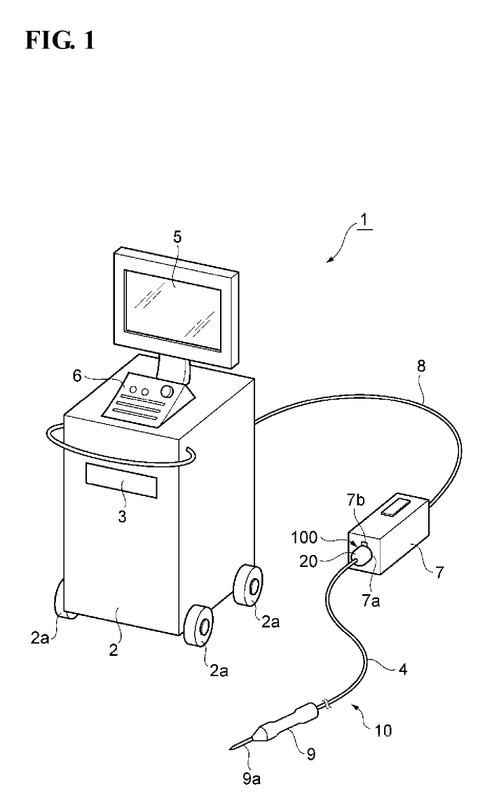

FIG. 1 is a conceptual view of a medical imaging system 1 for obtaining an optical coherence tomographic image. The medical imaging system 1 includes a console body portion 2, a rotational driving device 7, and an OCT catheter 10.

The console body portion 2 contains a light source, an optical measurement unit, a controller, a computer that performs image processing, a power supply, and the like, which realize the basic function for obtaining an internal body image. Casters 2a are attached to the console body portion 2 so that an operator can easily move the console body portion 2. A drive 3 into which a recording medium is inserted; a monitor 5 that displays an image; and input means 6, such as a keyboard and a mouse for accepting operational inputs, are disposed in or on an upper part of the console body portion 2.

The rotational driving device 7 is a device that performs a rotational scanning operation of the OCT catheter 10. The rotational driving device 7 is connected to the console body portion 2 via a cable 8, which includes electrical wiring and an optical fiber. The rotational driving device 7 is disposed at a position that is separated from the console body portion 2 and close to a patient.

The OCT catheter 10, which is connected to the rotational driving device 7, includes a hand piece 9 that a user grips when capturing an optical coherence tomographic image. The hand piece 9 is connected to a connection port (connection portion) 7a of the rotational driving device 7 via a connector structure at the distal end of a tube 4. In the present embodiment, when an outer casing 20 of a connector structure 100 of the OCT catheter 10 is pushed into a connection port 7a of the rotational driving device 7, the OCT catheter 10 is connected to the rotational driving device 7. A positioning portion 7b, for adjusting the position of the outer casing 20 of the OCT catheter 10 in the circumferential direction, is disposed at a peripheral edge of the connection port 7a. The positioning portion 7b in the example shown in the figure is a rectangular cutout that is formed at one position in the peripheral edge of the connection port 7a. A needle 9a is fixed to the distal end of the hand piece 9. The needle 9a rotatably contains a distal end portion of an optical fiber (not shown), and is configured so that light in the optical fiber can be emitted to the outside.

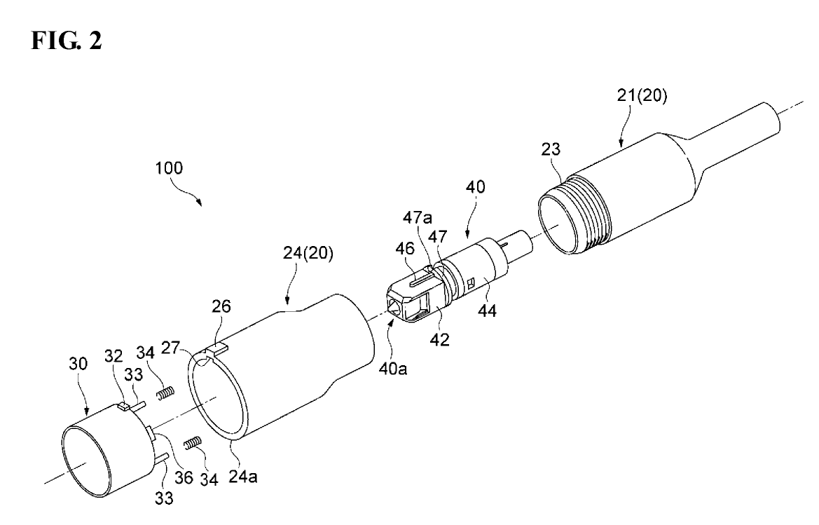

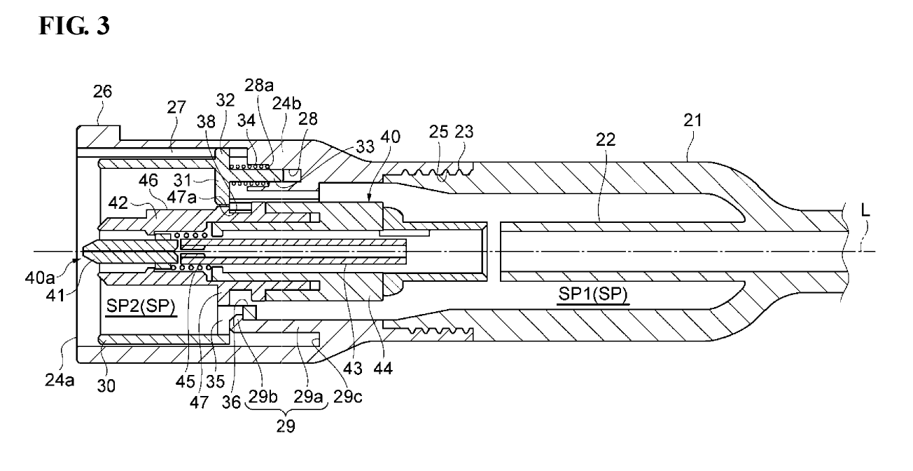

FIG. 2 is an exploded perspective view of the connector structure 100 of the OCT catheter 10. FIG. 3 is a sectional view of the connector structure 100 of the OCT catheter 10. As illustrated in FIGS. 2 and 3, the connector structure 100 of the OCT catheter 10 includes the outer casing 20, a stopper 30, and a connector 40. The outer casing 20 has an inner space SP extending in the axial direction L, and one end of the outer casing 20 in the axial direction L is open. The outer casing 20 of the present embodiment is composed of a rear shell 21 and a front shell 24. The rear shell 21, which is a rear part of the outer casing 20, has a substantially cylindrical shape whose diameter gradually decreases rearward. A tubular portion, into which a torque wire (not shown) is inserted, is formed in an inner space SP1 of the rear shell 21. A male thread 23, onto which the front shell 24 is to be screwed, is formed on a front end part of the rear shell 21. The tube 4 (see FIG. 1), which contains inner members such as the torque wire, is connected to the rear end of the rear shell 21.

The front shell 24 has a substantially cylindrical shape and has an inner space SP2 having a substantially circular cross-sectional shape. A female thread 25, which is to be screwed on the rear shell 21, is formed in a rear part of the front shell 24. The front shell 24 and the rear shell 21 form the outer casing 20 when the female thread 25 and the male thread 23 are screwed together. An end portion 24a on the front side of the front shell 24 is open. In the outer casing 20, the inner space SP2 of the front shell 24 and the inner space SP1 of the rear shell 21 are continuous from the opening in the end portion 24a. A positioning protrusion 26, which protrudes outward in the radial direction, is formed at one position on the outer surface of the front shell 24 in the circumferential direction. The outer casing 20 is inserted into the connection port 7a in a state in which the positions of the positioning portion 7b of the connection port 7a and the positioning protrusion 26 of the outer casing 20 in the circumferential direction coincide with each other.

In the inner surface of the front shell 24, a positioning groove 27, which extends rearward from the front end thereof in the axial direction L, is formed at one position in the circumferential direction. In the present embodiment, rotation of the stopper 30 relative to the outer casing 20 is restricted by the positioning groove 27. The position of the positioning groove 27 in the circumferential direction is substantially the same as the position of the positioning protrusion 26 in the circumferential direction.

A plurality of thick portions 24b, which extend inward in the radial direction, are formed on the inner surface of the front shell 24. A hole 28, which extends in the axial direction L and is open forward, is formed in each of the thick portions 24b. A step portion 28a is formed in the hole 28. The diameter of a front part the hole 28 on the front side (on the opening side) of the step portion 28a is larger than the diameter of a rear part of the hole 28 on the rear side of the step portion 28a. In the present embodiment, the holes 28 are formed at three positions in the circumferential direction with intervals of 120°.

A plurality of engagement hooks 29, which extend in the axial direction L, are formed in the inner space SP2 of the front shell 24. Each of the engagement hooks 29 includes a bar-shaped base portion 29a extending forward from a proximal end that is a step portion 29c, which extends inward in the radial direction from the inner surface of the front shell 24. A hook portion 29b, which protrudes inward in the radial direction, is formed at the front end of the base portion 29a. In the present embodiment, the engagement hooks 29 are formed at three positions in the circumferential direction with intervals of 120°. Each of the engagement hooks 29 is disposed between a corresponding pair of the holes 28 in the circumferential direction.

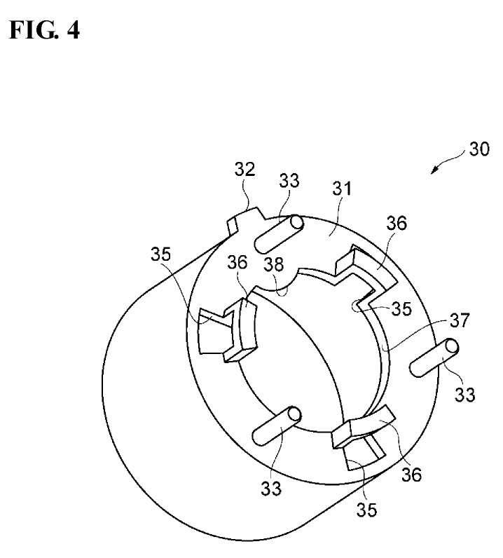

FIG. 4 is a perspective view of the stopper 30. As illustrated in FIGS. 2, 3, and 4, the stopper 30 is disposed in the inner space SP of the outer casing 20 and restricts rotation of the connector 40 relative to the outer casing 20. The stopper 30 has a substantially cylindrical shape and includes an annular stopper body 31 at the rear end thereof in the axial direction L. A positioning protrusion 32, which projects outward in the radial direction, is formed on the stopper body 31 at one position in the circumferential direction. The positioning protrusion 32 is contained in the positioning groove 27, which is formed in the inner surface of the front shell 24, and restricts rotation of the stopper 30 relative to the front shell 24.

A plurality of columnar shafts 33, which extend rearward in the axial direction L, are formed on the stopper body 31. The length of the shafts 33 is, for example, the same as the depth of the holes 28 of the front shell 24; and the diameter of the shafts 33 is smaller than the diameter of the rear parts of the holes 28. In the present embodiment, the shafts 33 are formed at three positions in the circumferential direction at intervals of 120°. In a state in which the stopper 30 is disposed in the inner space SP of the outer casing 20, the shafts 33 are inserted into springs 34, which are examples of an elastic member, and inserted into the holes 28. The outside diameter of the springs 34 is larger than the diameter of rear parts of the holes 28 and is smaller than the diameter of the front parts of the holes 28. Therefore, the springs 34 are disposed between the stopper body 31 and the step portions 28a. Thus, the stopper 30 is urged by the springs 34 forward in the axial direction L.

Cutout portions 35 and engagement tabs 36 are formed in the stopper body 31 at three positions in the circumferential direction so as to correspond to the engagement hooks 29. The base portions 29a of the engagement hooks 29 can be inserted into the cutout portions 35. The engagement tabs 36 are formed at inner parts, in the radial direction, of the cutout portions 35 so as to be engageable with the hook portions 29b of the engagement hooks 29. In the present embodiment, the engagement tabs 36 stand on the stopper body 31 so as to extend rearward in the axial direction L. In a state in which the engagement tabs 36 and the engagement hooks 29 are engaged with each other, the stopper 30 is prevented from coming off the outer casing 20. In a natural state, a state in which the engagement tabs 36 and the engagement hooks 29 are engaged with each other is maintained by the urging forces of the springs 34.

An engaging portion 38, which protrudes inward in the radial direction, is formed on an inner surface 37 of the stopper body 31. In the present embodiment, the inner periphery of the stopper body 31 has a substantially circular shape, and the engaging portion 38 is formed at one position in the circumferential direction. That is, the inside diameter of the stopper body 31 is reduced at the position in the circumferential direction where the engaging portion 38 is formed. The position of the engaging portion 38 in the circumferential direction is substantially the same as the position of the positioning protrusion 32 in the circumferential direction.

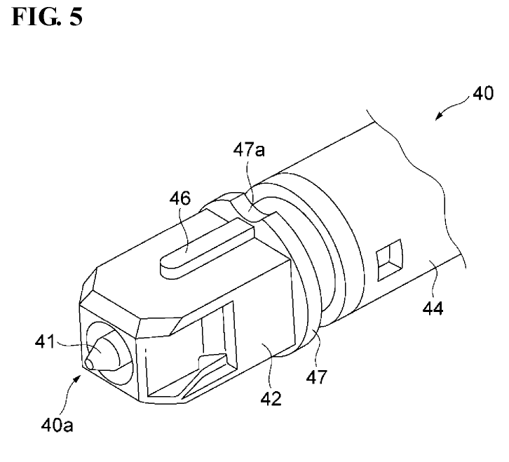

FIG. 5 is a perspective view of the connector 40. As illustrated in FIGS. 2, 3, and 5, the connector 40, which is a so-called SC connector, is disposed in the inner space SP of the outer casing 20 so as to be rotatable around the axis in a state in which a connection end 40a is oriented toward the opening. The connector 40 includes a ferrule 41, which holds an optical fiber, and a housing 42, which contains the ferrule 41. A cylindrical containing portion 44, which contains a joint 43 at the rear end of the ferrule 41, is disposed in a rear part of the housing 42. The joint 43 is connected, for example, to a torque wire (not shown). The optical fiber, which is held by the ferrule 41, rotates as the housing 42 rotates. In the example shown in the figures, a spring 45, which urges the ferrule 41 in the axial direction L, is disposed in the housing 42.

The housing 42 includes an engageable portion 47a, which is engageable with the engaging portion 38 of the stopper body 31. To be specific, a projection 46, which is used for positioning when connecting the connector to the adapter, is formed on the housing 42 of the present embodiment. A part of the housing 42 near the connection end 40a has a substantially rectangular cross-sectional shape, and the projection 46 protrudes outward. A rotation restricting portion 47, which has a substantially disc-like shape whose thickness direction is the axial direction L, is formed on the housing 42. The engageable portion 47a, which is recessed inward in the radial direction, is formed in the outer periphery of the rotation restricting portion 47. The outer periphery of the rotation restricting portion 47 of the housing 42 and the inner periphery of the stopper body 31 have the same shape. The thickness of the rotation restricting portion 47 in the axial direction L is substantially the same as the thickness of the stopper body 31 in the axial direction L.

In the axial direction L, the position of the rotation restricting portion 47 of the housing 42 is set so as to coincide with the position of the stopper body 31 in a state in which the engagement tabs 36 and the hook portions 29b are in contact with each other. Therefore, as illustrated in FIG. 3, in the state in which the engagement tabs 36 and the hook portions 29b are in contact with each other, the rotation restricting portion 47 can be disposed inside the inner periphery of the stopper body 31. In this state, the engageable portion 47a is engaged with the engaging portion 38, and therefore rotation of the connector 40 relative to the stopper 30 is restricted.

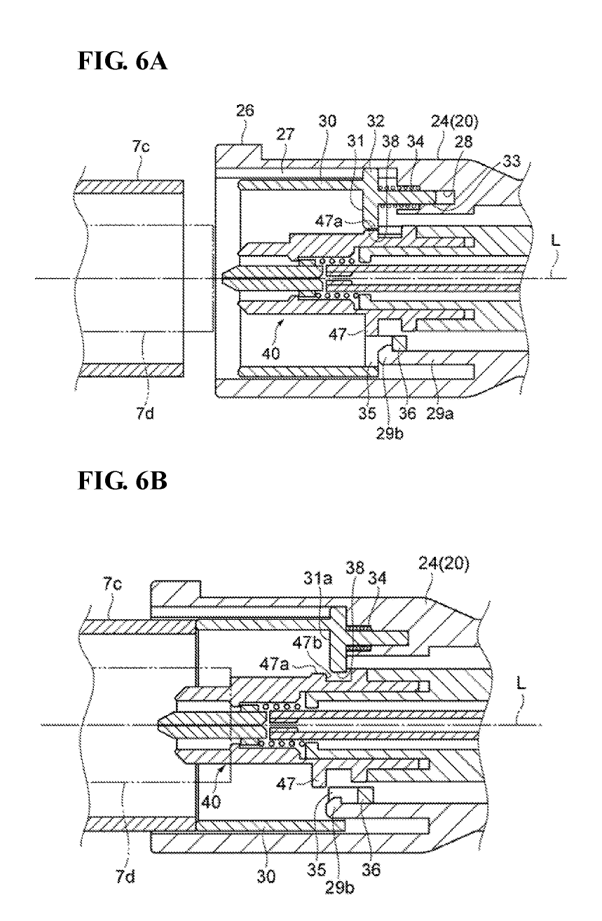

Next, referring to FIGS. 6A and 6B, an operation of the connector structure 100 of the OCT catheter 10 will be described. With the OCT catheter 10, when the end portion 24a of the outer casing 20 is pushed into the connection port 7a of the rotational driving device 7, the connector 40 is connected to an adapter 7d of the rotational driving device 7. FIG. 6A illustrates a state before the outer casing 20 is pushed into the connection port 7a of the rotational driving device 7. In FIGS. 6A and 6B, the housing of the rotational driving device 7 is omitted. Therefore, only a pressing portion 7c, which is fixed in the connection port 7a, is illustrated as the rotational driving device 7. The pressing portion 7c has, for example, a cylindrical shape. The pressing portion 7c can be inserted into the inner space SP of the outer casing 20 and has, for example, the same outside diameter as the stopper 30. The adapter 7d, which is to be connected to the connector 40, is disposed inside the pressing portion 7c. In the rotational driving device 7, the rotational position of the adapter 7d before the outer casing 20 is pushed corresponds to the rotational position of the connector 40 in a state in which rotation is restricted by the stopper 30.

The connector 40 is connected by pushing the outer casing 20 into the connection port 7a. At this time, the rotational position of the outer casing 20 is adjusted so that the positioning protrusion 26 of the outer casing 20 coincides with the positioning portion 7b of the connection port 7a. When the outer casing 20 is pushed into the connection port 7a, first, the rear end of the pressing portion 7c contacts the front end of the stopper 30. When the outer casing 20 is further pushed from this state, the stopper 30 is pressed rearward by the pressing portion 7c. The stopper 30 moves rearward relative to the outer casing 20 against the urging forces of the springs 34. At this time, the springs 34 contract due to elastic deformation. Because the position of the connector 40 relative to the outer casing 20 in the axial direction L does not change, the stopper 30 moves rearward also relative to the connector 40.

As illustrated in FIG. 6B, when a front end surface 31a of the stopper body 31 moves to a position behind a rear end surface 47b of the rotation restricting portion 47 in the axial direction L, the engaging portion 38 and the engageable portion 47a become disengaged. That is, restriction on rotation of the connector 40 by the stopper 30 is removed, and the connector 40 becomes rotatable. In this state, the connector 40 is connected to the adapter 7d and can rotate as the adapter 7d rotates.

The rotational position of the adapter 7d when the outer casing 20 is removed from the connection port 7a is controlled by the rotational driving device 7 so as to coincide with the rotational position of the adapter 7d before the outer casing 20 is pushed into the connection port 7a. Therefore, when the outer casing 20 is removed from the connection port 7a, rotation of the connector 40 is restricted by the stopper 30 again.

As described above, with the connector structure 100 of the OCT catheter 10 according to the present embodiment, because rotation of the connector 40 relative to the outer casing 20 is restricted by the stopper 30, the connector 40 does not rotate until the connector 40 is connected to the adapter 7d. In this case, a user can simultaneously change the rotational position of the connector 40 by rotating the outer casing 20 in the circumferential direction. That is, the rotational position of the connector 40 can be checked from the rotational position of the outer casing 20. On the other hand, when the outer casing 20 is pushed into the connection port 7a, restriction on the rotation of the connector 40 by the stopper 30 is removed. Thus, the connector 40 can be rotated by the rotational driving device 7. In this way, it is possible to connect the connector 40 and the adapter 7d easily without checking the rotational position of the connector 40 in the outer casing 20.

The mechanism for restricting rotation of the connector 40 by the stopper 30 includes the engaging portion 38 of the stopper 30 and the engageable portion 47a of the connector 40. When the outer casing 20 is pushed into the connection port 7a, the stopper 30 moves relative to the outer casing 20 in the axial direction L. Due to the relative movement of the stopper 30, the engaging portion 38 and the engageable portion 47a become disengaged. In this case, because the direction in which the outer casing 20 is pushed into the connection port 7a and the direction in which the stopper 30 moves are the same, a force used to push the outer casing 20 can be used to move the stopper 30.

Change in the position of the stopper 30 relative to the outer casing 20 is limited by the springs 34, each of which is an elastic member. Therefore, in a state in which the outer casing 20 is not pushed into the connection port 7a, the position of the stopper 30 relative to the outer casing 20 is maintained constant by the springs 34. Therefore, accidental removal of restriction on rotation of the connector 40, due to vibration or the like, is suppressed.

Rotation of the stopper 30 around the axis relative to the outer casing 20 is restricted. With such a structure, in a state in which rotation of the connector 40 is restricted, the rotational position of the connector 40 in the circumferential direction of the outer casing 20 is determined.

Heretofore, an embodiment of the present invention has been described with reference to the drawings. However, specific structures are not limited to those of the embodiment. For example, the front shell and the rear shell may be integrated with each other to form the outer casing. The outer casing may be segmented in the axial direction. The engaging portion (stopper body) and the engageable portion (rotation restricting portion) may only be in contact with each other in the circumferential direction to restrict rotational movement relative to each other. For example, the engaging portion may be recessed outward in the radial direction, and the engageable portion may protrude outward in the radial direction. For example, into a hole that is formed in one of the stopper body and the rotation restricting portion, a bar-shaped member formed in the other of the stopper body and the rotation restricting portion may be inserted. The inner shape of the stopper body and the outer shape of the rotation restricting portion may be the same shape that is not circular (for example, rectangular), and the stopper body and the rotation restriction portion may engage with each other. In the rotational driving device 7, it is not particularly necessary that the rotational position of the adapter when the outer casing is removed be controlled.

1. A connector structure of an OCT catheter to be connected to a rotational driving device that includes an adapter performing rotational driving, the connector structure comprising:
an outer casing that has an inner space extending in a direction of an axis and that has an opening at one end thereof in the direction of the axis;
a connector that is disposed in the inner space so as to be rotatable around the axis in a state in which a connection end thereof is oriented toward the opening; and
a stopper that is disposed in the inner space and that restricts rotation of the connector relative to the outer casing,
wherein, when a part of the outer casing including the one end is pushed into a connection portion of the rotational driving device, the connector is configured to be connectable to the adapter, and restriction on rotation of the connector by the stopper is removed.

2. The connector structure of an OCT catheter according to claim 1,
wherein a mechanism for restricting rotation of the connector by the stopper includes an engaging portion formed in the stopper and an engageable portion formed in the connector, the engageable portion being engageable with the engaging portion, and
wherein, when the outer casing is pushed into the connection portion, the stopper moves relative to the outer casing in a direction that is the direction of the axis and in which the stopper is pushed, and the engaging portion and the engageable portion are configured to disengage due to relative movement of the stopper.

3. The connector structure of an OCT catheter according to claim 12, wherein
change in position of the stopper relative to the outer casing is limited by an elastic member, and
wherein, when the outer casing is pushed into the connection portion, a position of the stopper relative to the outer casing is changed while the elastic member is being deformed, and restriction on rotation of the connector by the stopper is removed.

4. The connector structure of an OCT catheter according to claim 1,
wherein rotation of the stopper relative to the outer casing around the axis is restricted.

5. The connector structure of an OCT catheter according to claim 1,
wherein the stopper includes an engaging portion and is configured to move in the direction of the axis relative to the outer casing, and
wherein the connector includes an engageable portion that is engageable with the engaging portion, and the connector is configured so that, when the stopper moves relative to the outer casing in a direction that is the direction of the axis and in which the stopper is pushed, the engaging portion and the engageable portion are configured to disengage and the connector is configured to rotate relative to the stopper.

6. The connector structure of an OCT catheter according to claim 1,
wherein the connector structure of an OCT catheter includes an elastic member that is disposed between the outer casing and the stopper and that is configured to extend or contract in the direction of the axis,
wherein, when the elastic member extends and the stopper moves relative to the outer casing in a direction that is the direction of the axis and in which the stopper is pulled out, rotation of the connector is restricted, and
wherein, when the elastic portion contracts and the stopper moves relative to the outer casing in a direction that is the direction of the axis and in which the stopper is pushed, restriction on rotation is removed.

7. The connector structure of an OCT catheter according to claim 1,
wherein the outer casing includes a positioning groove in an inner surface thereof that defines the inner space, the positioning groove being parallel to the axis, and
wherein the stopper includes a positioning protrusion that engages with the positioning groove.

8. A medical imaging system comprising the connector structure of an OCT catheter according to claim 1 and the rotational driving device,
wherein the medical imaging system is controlled so that a rotational position of the adapter of the rotational driving device when the outer casing is pushed into the connection portion coincides with a rotational position of the adapter when the outer casing is removed from the connection portion.
